(19) United States
(12) Patent Application Publication
(10) Pub. No.: US 2026/0334810 A1
(43) Pub. Date: Oct. 29, 2026

(54) CLOSED-LOOP SYSTEM TO TERMINATE APNEIC EVENTS AND SNORING

(71) Applicant: MAKANI SCIENCE INC., Irvine, CA (US)

(72) Inventors: Michael Chu, Irvine, CA (US); Greg Buchert, Irvine, CA (US)

(21) Appl. No.: 19/649,700

(22) Filed: Apr. 16, 2026

Related U.S. Application Data
(60) Provisional application No. 63/796,631, filed on Apr. 29, 2025.

(51) Int. Cl. A61B 5/08; A61B 5/00; A61B 5/11; A61B 5/113; A61B 5/1455; A61B 5/28

(52) U.S. Cl. CPC A61B 5/0826; A61B 5/0803; A61B 5/1126; A61B 5/113; A61B 5/14551; A61B 5/28; A61B 5/4836; A61B 5/6823; A61B 5/6833; A61B 5/721; A61B 5/7264; A61B 5/7275; A61B 2503/04; A61B 2562/0204; A61B 2562/0219; A61B 2562/0261; A61B 2562/164

(57) ABSTRACT

A system for respiratory event prediction and detection in a patient. The system includes a multimodal sensor system having a plurality of sensor devices including mechanical sensors, configured to generate one or more output signals based on the body of the patient. The system further includes a computing device communicatively coupled to the multimodal sensor system, having a predictive model configured to accept the one or more output signals and generate an output including an identification of a respiratory event, a prediction of a future respiratory event, or a combination thereof.

### FIELD OF THE INVENTION

This application is a non-provisional and claims benefit of U.S. Provisional Application No. 63/796,631 filed Apr. 29, 2025, the specification of which is incorporated herein in its entirety by reference.

### FIELD OF THE INVENTION

The present invention is directed to systems for identifying patterns in respiratory events based on mechanical signals, light-based signals, electrical signals, acoustic signals, or a combination thereof and subsequent categorization and interruption of said respiratory events (e.g. apnea, snoring).

### BACKGROUND OF THE INVENTION

Apnea is a common condition in individuals of all ages that has multiple etiologies. Apnea of prematurity (AOP) is a common newborn condition defined as a temporary cessation of breathing. Predominantly affecting premature infants, its incidence is inversely correlated with gestational age, with nearly 100% occurrence in infants born prior to the 29th week of gestation. While AOP generally resolves with infant maturation, the consequence of missed or frequent apneic episodes can be severe, ranging from acute cardiorespiratory failure to long-term neurological impairment.

The current standard for apnea monitoring relies on conventional vital sign monitors to notify healthcare professionals (HCPs) when an apnea event occurs; it is then the HCP's responsibility to respond and document the apnea event. While this is the current practice, there are multiple downsides to this approach. First, manual documentation of apnea is often inaccurate, with reports indicating that HCP documentation missed between 50% to 70% of apnea cases compared to continuous monitoring. Furthermore, HCPs are frequently overextended and may not respond immediately to alarms, a factor contributed to by alarm fatigue, a common issue in neonatal intensive care units (NICU). Since documentation is one of the parameters used to determine patient discharge, inaccurate reporting may put the newborn at additional risk. In home environments, HCPs are not present and people suffering from apnea events are at a much higher risk due to the lack of options for detection and treatment of these events outside of hospital settings.

Second, current vital monitors can only detect apnea, and not differentiate the three types of apnea events (central, obstructive and mixed apnea) common with AOP. Unless a nurse or doctor is physically present to witness obstructive or mixed apnea events, these types of apneas are likely to be overlooked. More importantly, the treatments for central and obstructive apneas are different due to their distinct pathology; thus, improved understanding and identification of AOP will provide optimal treatments for the patients and potentially reduce complications.

Given the current standard for monitoring AOP, there is a significant need for an accurate and continuous apnea monitor that can identify and differentiate between central, obstructive, and mixed apnea. Such a device will not only improve safety for the newborn but will also help provide better care, reducing the financial burden on the family and the workload for the hospital.

There are three primary sensor technologies that can identify AOP. While all are commonly found in healthcare, they are ill-suited for a newborn population. Flow monitoring technology directly measures airflow from the mouth/nose of the patient but is limited to newborns who are intubated. Impedance monitoring sensors measure the change in electrical impedance across the torso (from breathing) and are commonly implemented with standard vital monitors. While both flow and impedance monitors are in the NICU, these technologies are only sensitive to central apnea. Respiratory Inductive Plethysmography (RIP) uses two belts to monitor the circumferential change of the abdomen and chest wall from respiration. Unlike flow and impedance sensors, RIP can distinguish the difference between central and obstructive apnea. However, the bulky form factor and high sensitivity to motion make it a poor monitor for newborns.

AOP is a prevalent condition, yet consensus on best practices and care remains elusive, likely due to limitations in monitoring technology. The monitors used in research and the NICU are large and bulky, hindering accessibility and patient comfort. Consequently, this limits the ability to collect high-quality data, thereby constraining research efforts (e.g., development of machine learning tools). While there are notable successes in automatically identifying apnea in both adults and newborns, the research relies on inaccessible monitors and a relatively small patient population. Considering a diverse and comprehensive dataset is required to better understand AOP, data collection is of utmost importance and can be expedited by an accessible, respiratory-specific monitor. Such a system would provide researchers with a streamlined tool for collecting information on AOP and other conditions. Moreover, integrating additional clinical metrics (e.g., patient treatment, condition) will help HCPs determine the most optimal treatments for improving their patient's outcomes.

### BRIEF SUMMARY OF THE INVENTION

It is an objective of the present invention to provide systems that allow for identifying patterns in respiratory events based on mechanical signals and subsequent categorization and interruption of said respiratory events (e.g. apnea, snoring), as specified in the independent claims. Embodiments of the invention are given in the dependent claims. Embodiments of the present invention can be freely combined with each other if they are not mutually exclusive.

The present invention features a system for respiratory event prediction and detection in a patient. The system may comprise a multimodal sensor system comprising one or more sensor devices disposed on or proximate to a body of the patient, configured to generate a plurality of output signals. The one or more sensor devices may comprise a plurality of mechanical sensors disposed on a torso of the patient configured to measure one or more mechanical properties of the patient and generate one or more respiratory signals based on the one or more mechanical properties. The plurality of output signals may comprise the one or more respiratory signals. The system may further comprise a computing device communicatively coupled to the multimodal sensor system. The computing device may comprise a predictive model configured to accept the plurality of output signals and generate an output comprising an identification of a respiratory event, a prediction of a future respiratory event, or a combination thereof.

One of the unique and inventive technical features of the present invention is the implementation of an array of mechanical strain sensors disposed on or proximate to a body of a patient. The strain sensors are placed on the body at various orientations and are linked to one or more devices that send the sensor data via bluetooth to the monitor. Each sensor is integrated in a soft patch, which is then integrated with the hardware. The sensor can be placed horizontally on the chest (left and/or right side), vertically on the chest (left and/or right side), angled on the chest (left and/or right side), vertical on the back (left and/or right side), horizontal on the back (left and/or right side), angled on the back (left and/or right side), horizontal on the abdomen (left and/or right side), vertical on the abdomen (left and/or right side), and/or angled on the abdomen (left and/or right side). Without wishing to limit the invention to any theory or mechanism, it is believed that the technical feature of the present invention advantageously provides for efficient and accurate detection of respiratory events in a patient, allowing for compatibility with infants. None of the presently known prior references or work has the unique inventive technical feature of the present invention.

Furthermore, the inventive technical features of the present invention contributed to a surprising result. For example, prior respiratory monitoring systems implement a single RIP belt or a single bioimpedance sensor to minimize patient discomfort. One of ordinary skill in the art would expect that the introduction of more sensors would not only increase patient discomfort but restrict the type of patient compatible with the system to adults. Surprisingly, the presently claimed invention is able to achieve a comfortable sensor system compatible with patients of any age and size through the use of mechanical sensors primarily. Thus, the inventive technical feature of the present invention contributed to a surprising result.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWINGS

The features and advantages of the present invention will become apparent from a consideration of the following detailed description presented in connection with the accompanying drawings in which:

FIG. 1A shows a schematic diagram of the sensor system of the present invention.

FIG. 1B shows a schematic diagram of the sensor system of the present invention worn by an infant patient.

FIG. 2 shows respiratory waveforms from the ribcage and abdominal sensors of an embodiment of the system of the present invention.

FIG. 3 shows preliminary data showing unique respiratory waveform patterns captured by the strain sensors of the present invention.

FIG. 4 shows example features that can be extracted from the strain sensor waveforms gathered by the system of the present invention.

FIGS. 5A-5B show example high-level embodiments of the sensor devices of the present invention.

FIGS. 6A-6C show views of alternate embodiments of the sensor devices of the present invention.

FIG. 7 shows a patch component diagram of an embodiment of the sensor device of the present invention.

FIG. 8 shows a trace component diagram of an embodiment of the sensor device of the present invention.

FIG. 9 shows an embodiment of a patch stack of the sensor device of the present invention.

FIGS. 10A-10B show alternate embodiment of the patch stack of the sensor device of the present invention.

FIG. 11 shows a chart of each system component and the flow of data from the wearable device to the monitor display.

FIG. 12 shows a chart showing how data is moved through the system. The lines and arrows show the direction of data flow. The data is combined into a single stream to be sent via Bluetooth.

FIG. 13 shows a flowchart for using the NMS.

FIG. 14 shows a side profile of the enclosure and patch interface.

FIG. 15A shows a table of components that may contact the patient and their material.

FIG. 15B shows a table of components that will not contact the patient and their material.

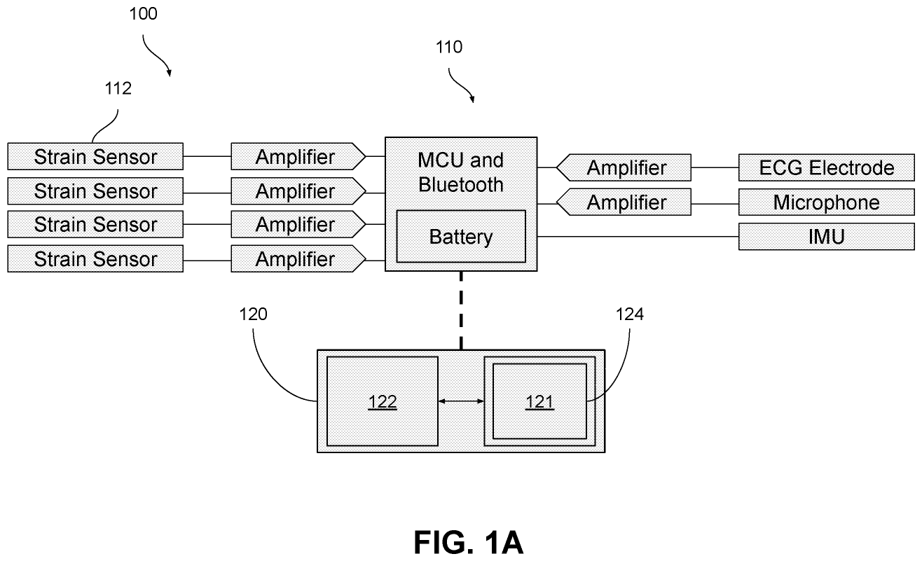

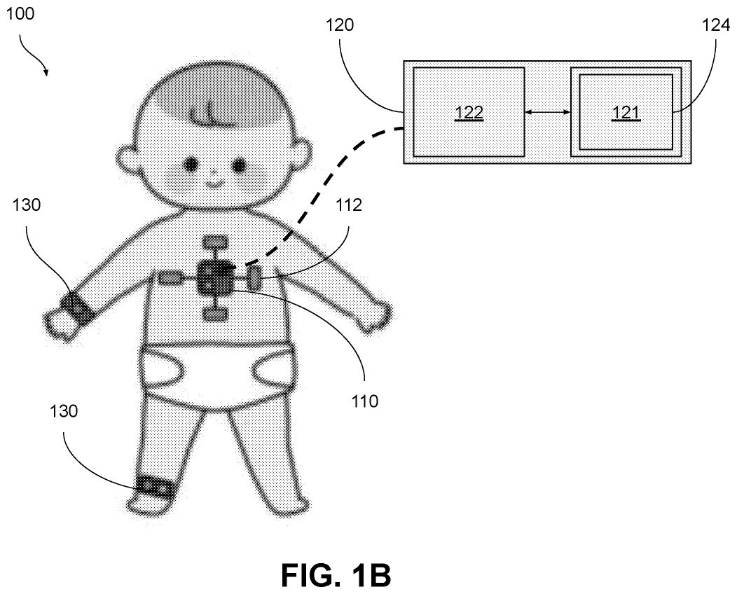

### DETAILED DESCRIPTION OF THE INVENTION

Following is a list of elements corresponding to a particular element referred to herein:
system 100
multimodal sensor system 110
mechanical sensors 112
computing device 120
predictive model 121
processor 122
memory component 124
stimulator 130

The term “torso” is defined herein as the chest, abdomen, and back of a person's body.

The term “body” is defined herein as any physical part of a person, such as the torso, appendages, or head of a person.

The term “infant” is defined herein as any human being 2 years of age or less.

In some aspects, the techniques described herein relate to a system (100) for respiratory event prediction and detection in a patient, the system (100) including: a. a multimodal sensor system (110) including a plurality of sensor devices disposed on or proximate to a body of the patient, configured to generate one or more output signals, wherein the plurality of sensor devices includes a plurality of mechanical sensors (112) disposed on a torso of the patient configured to measure one or more mechanical properties of the patient and generate one or more respiratory signals based on the one or more mechanical properties, wherein the one or more output signals includes the one or more respiratory signals, wherein the plurality of mechanical sensors (112) include soft electronic strain sensors; and b. a computing device (120) communicatively coupled to the multimodal sensor system (110), including a software model (121) configured to accept the one or more output signals and generate an output including an identification of a respiratory event, a prediction of a future respiratory event, or a combination thereof.

In some aspects, the techniques described herein relate to a system (100), wherein the plurality of mechanical sensors (112) further include motion sensors, internal measurement units (IMUs), or a combination thereof.

In some aspects, the techniques described herein relate to a system (100), wherein the plurality of sensor devices further include: a. one or more electrical sensors configured to measure one or more electrical signals; b. one or more acoustic sensors configured to measure one or more acoustic signals; c. one or more light sensors configured to measure one or more light-based signals; and d. one or more chemical sensors configured to measure one or more chemical signals.

In some aspects, the techniques described herein relate to a system (100), wherein the predictive model (121) is further configured to filter one or more false positives from the output based on the one or more electrical signals, the one or more acoustic signals, the one or more light-based signals, the one or more chemical signals, or a combination thereof.

In some aspects, the techniques described herein relate to a system (100), wherein the plurality of mechanical sensors (112) further include accelerometers configured to measure one or more internal motion signals, one or more external motion signals, or a combination thereof, wherein the predictive model (121) is further configured to filter one or more motion artifacts from the output based on the one or more internal motion signals, the one or more external motion signals, or the combination thereof.

In some aspects, the techniques described herein relate to a system (100) further including a stimulator device (130) communicatively coupled to the computing device (120) and disposed on or proximate to the body of the patient, configured to apply stimulation to the body of the patient in response to the identification of the respiratory event, the prediction of the future respiratory event, or the combination thereof such that the respiratory event, the future respiratory event, or a combination thereof is interrupted.

In some aspects, the techniques described herein relate to a system (100), wherein the stimulation applied by the stimulator device (130) includes vibration, heat, sound, electrical current, movement, or a combination thereof.

In some aspects, the techniques described herein relate to a system (100), wherein the respiratory event, the future respiratory event, or the combination thereof includes apnea, snoring, external obstruction of breathing, internal obstruction of breathing, Sudden Infant Death Syndrome (SIDS), or a combination thereof.

In some aspects, the techniques described herein relate to a system (100), wherein the predictive model (121) includes an algorithm, a machine learning model, or a combination thereof.

In some aspects, the techniques described herein relate to a system (100) for apneic event prediction and detection in a patient, the system (100) including: a. a multimodal sensor system (110) including a plurality of sensor devices disposed on or proximate to a body of the patient, configured to generate e output signals, wherein the plurality of sensor devices includes a plurality of mechanical sensors (112) disposed on a torso of the patient configured to measure one or more mechanical properties of the patient and generate one or more respiratory signals based on the one or more mechanical properties, wherein the one or more output signals includes the one or more respiratory signals, wherein the plurality of mechanical sensors (112) include soft electronic strain sensors; b. a computing device (120) communicatively coupled to the multimodal sensor system (110), including a predictive model (121) configured to: i. accept the one or more output signals; ii. generate an output including an identification of an apneic event, a prediction of a future apneic event, or a combination thereof; iii. categorize the apneic event, the future apneic event, or a combination thereof as central apnea, obstructive apnea, or a combination thereof; and iv. transmit a signal to a stimulator device (130) in response to the identification of the apneic event, the prediction of the future apneic event, or the combination thereof; and c. the stimulator device (130) communicatively coupled to the computing device (120) and disposed on the body of the patient, configured to apply stimulation to the body of the patient in response to the identification of the apneic event, the prediction of the future apneic event, or the combination thereof such that the apneic event, the future apneic event, or the combination thereof is interrupted.

In some aspects, the techniques described herein relate to a system (100), wherein the plurality of mechanical sensors (112) further include motion sensors, internal measurement units (IMUs), or a combination thereof.

In some aspects, the techniques described herein relate to a system (100), wherein the plurality of sensor devices further include: a. one or more electrocardiogram (ECG) electrodes configured to measure one or more electrical activity signals; b. one or more microphones configured to measure one or more sound signals; c. one or more internal measurement units (IMUs) configured to measure one or more internal motion signals; d. one or more pulse oximeter devices configured to measure one or more blood oxygenation signals; and e. one or more motion sensors configured to measure one or more external motion signals.

In some aspects, the techniques described herein relate to a system (100), wherein the predictive model (121) is further configured to filter one or more false positives from the output based on the one or more electrical activity signals, the one or more sound signals, the one or more blood oxygenation signals, or a combination thereof.

In some aspects, the techniques described herein relate to a system (100), wherein the predictive model (121) is further configured to filter one or more motion artifacts from the output based on the one or more internal motion signals, the one or more external motion signals, or a combination thereof.

In some aspects, the techniques described herein relate to a system (100), wherein the stimulation applied by the stimulator device (130) includes vibration, heat, sound, electrical current, or a combination thereof.

In some aspects, the techniques described herein relate to a system (100), wherein the future apneic event includes apnea prematurity.

In some aspects, the techniques described herein relate to a system (100), wherein categorizing the apneic event, the future apneic event, or the combination thereof includes determining whether the apneic event, the future apneic event, or the combination thereof includes central apnea, obstructive apnea, or a combination thereof.

In some aspects, the techniques described herein relate to a system (100), wherein the predictive model (121) includes an algorithm, a machine learning model, or a combination thereof.

In some aspects, the techniques described herein relate to a system (100) for apneic event prediction and detection in an infant patient, the system (100) including: a. a multimodal sensor system (110) including a plurality of sensor devices disposed on or proximate to a body of the infant patient, configured to generate one or more output signals, wherein the plurality of sensor devices includes a plurality of mechanical sensors (112) disposed on a torso of the infant patient configured to measure one or more mechanical properties of the infant patient and generate one or more respiratory signals based on the one or more mechanical properties, wherein the one or more output signals includes the one or more respiratory signals, wherein the plurality of mechanical sensors (112) further include soft electronic strain sensors; b. a computing device (120) communicatively coupled to the multimodal sensor system (110), including a processor (122) configured to execute computer-readable instructions and a memory component (124) operatively coupled to the processor (122), including: i. a predictive model (121) configured to accept the one or more output signals and generate an output including an identification of an apneic event, a prediction of a future apneic event, or a combination thereof; and ii. computer-readable instructions for: A. inputting the one or more output signals into the predictive model (121) such that the predictive model (121) generates the output; B. determining, based on the output, whether the apneic event, the future apneic event, or the combination thereof includes central apnea, obstructive apnea, or a combination thereof; and C. transmitting a signal to a stimulator device (130) in response to the identification of the apneic event, the prediction of the future apneic event, or the combination thereof; and c. the stimulator device (130) communicatively coupled to the computing device (120) and disposed on the body of the infant patient, configured to apply stimulation to the body of the infant patient in response to the signal from the computing device (120) such that the apneic event, the future apneic event, or the combination thereof is interrupted.

In some aspects, the techniques described herein relate to a system (100), wherein the predictive model (121) includes an algorithm, a machine learning model, or a combination thereof.

In some embodiments, the plurality of mechanical sensors (112) may comprise any soft elastomeric electronic sensors. In some embodiments, the computing device (120) may comprise a cloud server with which the multimodal sensor system (110) is able to connect to through a wireless connection. In some embodiments, the one or more electrical sensors may be configured to generate one or more electrical signals comprising a pulse oximetry signal, an electrocardiogram (ECG) signal, or a combination thereof. In some embodiments, the plurality of mechanical sensors (112) may comprise one or more motion sensors. The motion artifacts from the motion sensors may be used to calculate motion alongside the motion signals themselves.

The present invention features a system (100) for respiratory event prediction and detection in a patient. In some embodiments, the system (100) may comprise a multimodal sensor system (110) comprising one or more sensor devices disposed on or proximate to a body of the patient, configured to generate a plurality of output signals. The one or more sensor devices may comprise a plurality of mechanical sensors (112) disposed on a torso of the patient configured to measure one or more mechanical properties of the patient and generate one or more respiratory signals based on the one or more mechanical properties. The plurality of output signals may comprise the one or more respiratory signals. The system (100) may further comprise a computing device (120) communicatively coupled to the multimodal sensor system (110). The computing device (120) may comprise a predictive model (121) configured to accept the plurality of output signals and generate an output comprising an identification of a respiratory event, a prediction of a future respiratory event, or a combination thereof.

In some embodiments, the plurality of mechanical sensors (112) may comprise strain sensors, motion sensors, internal measurement units (IMUs), or a combination thereof. In some embodiments, the one or more sensor devices may further comprise one or more electrocardiogram (ECG) electrodes configured to measure one or more electrical activity signals, one or more microphones configured to measure one or more sound signals, one or more internal measurement units (IMUs) configured to measure one or more internal motion signals, one or more pulse oximeter devices configured to measure one or more blood oxygenation signals, and one or more motion sensors configured to measure one or more external motion signals. In some embodiments, the predictive model (121) may be further configured to filter one or more false positives from the output based on the one or more electrical activity signals, the one or more sound signals, the one or more blood oxygenation signals, or a combination thereof. In some embodiments, the predictive model (121) may be further configured to filter one or more motion artifacts from the output based on the one or more internal motion signals, the one or more external motion signals, or a combination thereof.

The system (100) may further comprise a stimulator device (130) communicatively coupled to the computing device (120) and disposed on the body of the patient, configured to apply stimulation to the body of the patient in response to the identification of the respiratory event, the prediction of the future respiratory event, or the combination thereof such that the respiratory event, the future respiratory event, or a combination thereof is interrupted. In some embodiments, the stimulation applied by the stimulator device (130) may comprise vibration, heat, sound, electrical current, or a combination thereof. For example, the stimulation may comprise a bed configured to raise such that the patient's position is adjusted in response to snoring (e.g., a SleepNumber^{®} bed). In some embodiments, the respiratory event, the future respiratory event, or the combination thereof may comprise apnea, snoring, external obstruction of breathing, internal obstruction of breathing, Sudden Infant Death Syndrome (SIDS), seizures, or a combination thereof. In some embodiments, the predictive model (121) may comprise an algorithm, a machine learning model, or a combination thereof.

The present invention features a system (100) for apneic event prediction and detection in a patient. In some embodiments, the system (100) may comprise a multimodal sensor system (110) comprising one or more sensor devices disposed on or proximate to a body of the patient, configured to generate a plurality of output signals. The one or more sensor devices may comprise a plurality of mechanical sensors (112) disposed on a torso of the patient configured to measure one or more mechanical properties of the patient and generate one or more respiratory signals based on the one or more mechanical properties. The plurality of output signals may comprise the one or more respiratory signals.

The system (100) may further comprise a computing device (120) communicatively coupled to the multimodal sensor system (110). The computing device (120) may comprise a predictive model (121) configured to accept the plurality of output signals and generate an output comprising an identification of an apneic event, a prediction of a future apneic event, or a combination thereof, and categorize the apneic event, the future apneic event, or a combination thereof. The system (100) may further comprise a stimulator device (130) communicatively coupled to the computing device (120) and disposed on or proximate to the body of the patient, configured to apply stimulation to the body of the patient in response to the identification of the apneic event, the prediction of the future apneic event, or the combination thereof such that the apneic event, the future apneic event, or the combination thereof is interrupted.

In some embodiments, the mechanical sensors (112) may comprise strain sensors, motion sensors, internal measurement units (IMUs), or a combination thereof. In some embodiments, the one or more sensor devices may further comprise one or more electrocardiogram (ECG) electrodes configured to measure one or more electrical activity signals, one or more microphones configured to measure one or more sound signals, one or more internal measurement units (IMUs) configured to measure one or more internal motion signals, one or more pulse oximeter devices configured to measure one or more blood oxygenation signals, and one or more motion sensors configured to measure one or more external motion signals. In some embodiments, the predictive model (121) may be further configured to filter one or more false positives from the output based on the one or more electrical activity signals, the one or more sound signals, the one or more blood oxygenation signals, or a combination thereof. In some embodiments, the predictive model (121) may be further configured to filter one or more motion artifacts from the output based on the one or more internal motion signals, the one or more external motion signals, or a combination thereof.

In some embodiments, the one or more sensor devices may comprise one or more electrical sensors configured to measure one or more electrical signals. In some embodiments, the one or more sensor devices may comprise one or more acoustic sensors configured to measure one or more acoustic signals. In some embodiments, the one or more sensor devices may comprise one or more light sensors configured to measure one or more light-based signals (e.g. photoplethysmography). In some embodiments, the one or more sensor devices may comprise one or more chemical sensors configured to measure one or more chemical signals. In some embodiments, the chemical signals may comprise the detection of sweat.

In some embodiments, the stimulation applied by the stimulator device (130) may comprise vibration, heat, sound, electrical current, or a combination thereof. In some embodiments, the future apneic event may comprise apnea of prematurity. In some embodiments, categorizing the apneic event, the future apneic event, or the combination thereof may comprise determining whether the apneic event, the future apneic event, or the combination thereof comprises central apnea, obstructive apnea, or a combination thereof. In some embodiments, the predictive model (121) may comprise an algorithm, a machine learning model, or a combination thereof. In some embodiments, the stimulator device (130) may comprise a motorized mechanism operatively coupled to a bed of the patient, configured to change the bed position in response to signals from the multimodal sensor system (110). In some embodiments, the stimulator device (130) may comprise a speaker device, configured to play soothing audio (i.e. calming sounds, calming music, the voice of the mother) in response to signals from the multimodal sensor system (110). In some embodiments, the stimulator device (130) of the present invention may be used to prevent or interrupt snoring during sleep.

The present invention features a system (100) for apneic event prediction and detection in an infant patient. In some embodiments, the system (100) may comprise a multimodal sensor system (110) comprising one or more sensor devices disposed on or proximate to a body of the infant patient, configured to generate a plurality of output signals. The one or more sensor devices may comprise a plurality of mechanical sensors (112) disposed on a torso of the infant patient configured to measure one or more mechanical properties of the infant patient and generate one or more respiratory signals based on the one or more mechanical properties. The plurality of output signals may comprise the one or more respiratory signals.

The system (100) may further comprise a computing device (120) communicatively coupled to the multimodal sensor system (110), comprising a processor (122) configured to execute computer-readable instructions and a memory component (124) operatively coupled to the processor (122). The memory component (124) may comprise a predictive model (121) configured to accept the plurality of output signals and generate an output comprising an identification of an apneic event, a prediction of a future apneic event, or a combination thereof. The memory component (124) may further comprise computer-readable instructions for inputting the plurality of output signals into the predictive model (121) such that the predictive model (121) generates the output, determining, based on the output, whether the apneic event, the future apneic event, or the combination thereof comprises central apnea, obstructive apnea, or a combination thereof, and transmitting a signal to a stimulator device (130) in response to the identification of the apneic event, the prediction of the future apneic event, or the combination thereof.

The system (100) may further comprise the stimulator device (130) communicatively coupled to the computing device (120) and disposed on the body of the infant patient, configured to apply stimulation to the body of the infant patient in response to the signal from the computing device (120) such that the apneic event, the future apneic event, or the combination thereof is interrupted. In some embodiments, the predictive model (121) may comprise an algorithm, a machine learning model, or a combination thereof.

In some embodiments, the one or more mechanical properties measured by the mechanical sensors of the present invention may comprise chest wall expansion, chest wall contraction, abdomen expansion, and abdomen contraction. The sensors may be configured to generate respiratory signals based on the mechanical properties, comprising respiratory rate, volume (depth), flow (change in slope of the waveform), absence of waveform, inhalation time, exhalation time, inhalation amplitude, exhalation amplitude, and phase shift between the different mechanical sensor data. In some embodiments, the present invention may implement a predictive model. The training dataset for the predictive models may comprise raw respiration waveforms, raw pulse oximeter waveforms, raw ECG waveforms, raw accelerometer waveforms, raw sound waveforms, calculated respiratory rate, respiratory depth, slope, exhalation time, inhalation time, inhalation amplitude, exhalation amplitude, respiration phase shift, heart rate, blood oxygenation (relative), motion, etc. The predictive model may comprise a random forest, support vector machine, Gradient boosting, k-nearest neighbors, 1D convolutional neural network, etc. If the present invention identifies an apnea event, an output may be produced in the form of a visual and audio alert of the apnea event, as well as vital sign signals corresponding to the apnea event. Furthermore, if an apnea event is detected, the stimulator may activate. This could occur when the event is predicted or at the onset of the event. Another example is if snoring is detected, a bed embodiment of the stimulator may adjust itself upward to clear the airway and reduce snoring.

### EXAMPLE

The following is a non-limiting example of the presently claimed invention.

Apnea of prematurity (AOP) is a common newborn condition defined as a temporary cessation of breathing. Predominantly affecting premature infants, its incidence is inversely correlated with gestational age, with nearly 100% occurrence in infants born prior to the 29th week of gestation. While AOP generally resolves with infant maturation, the consequence of missed or frequent apneic episodes can be severe, ranging from acute cardiorespiratory failure to long term neurological impairment.

The current standard for apnea monitoring relies on conventional vital sign monitors to notify healthcare professionals (HCPs) when an apnea event occurs; it is then the HCP's responsibility to respond and document the apnea event. While this is the current practice, there are multiple downsides to this approach. First, manual documentation of apnea is often inaccurate, with reports indicating that HCP documentation missed between 50% to 70% of apnea cases compared to continuous monitoring. Furthermore, HCPs are frequently overextended and may not respond immediately to alarms, a factor contributed to by alarm fatigue, a common issue in neonatal intensive care units (NICU). Since documentation is one of the parameters used to determine patient discharge, inaccurate reporting may put the newborn at additional risk.

Second, current vital monitors can only detect central apnea, missing the other two apnea events (obstructive and mixed apnea) common with AOP. Unless a nurse or doctor is physically present to witness obstructive or mixed apnea events, these types of apneas are likely to be overlooked. More importantly, the treatment for central and obstructive apneas are different due to their distinct pathology; thus, improved understanding and identification of AOP provides optimal treatments for the patients and potentially reduce complications.

Given the current standard for monitoring AOP, there is a significant need for an accurate and continuous apnea monitor that can identify and differentiate between central, obstructive, and mixed apnea. Such a device not only improves safety for the newborn but also helps provide better care, reducing the financial burden on the family and the workload for the hospital.

There are three primary sensor technologies that can identify AOP. While all are commonly found in healthcare, they are ill-suited for a newborn population. Flow monitoring technology directly measures airflow from the mouth/nose of the patient but is limited to newborns who are intubated. Impedance monitoring sensors measure the change in electrical impedance across the torso (from breathing) and are commonly implemented with standard vital monitors. While both flow and impedance monitors are in the NICU, these technologies are only sensitive to central apnea. Respiratory Inductive Plethysmography (RIP) uses two belts to monitor the circumferential change of the abdomen and chest wall from respiration. Unlike flow and impedance sensors, RIP can distinguish the difference between central and obstructive apnea. However, the bulky form factor and high sensitivity to motion makes it a poor monitor for newborns.

AOP is a prevalent condition, yet consensus on best practices and care remains elusive, likely due to limitations in monitoring technology. The monitors used in research and the NICU are large and bulky, hindering accessibility and patient comfort. Consequently, this limits the ability to collect high quality data, thereby constraining research efforts (e.g., development of machine learning tools). While there are notable successes in automatically identifying apnea in both adults and newborns, the research relies on inaccessible monitors and a relatively small patient population. Considering a diverse and comprehensive dataset is required to better understand AOP, data collection is of utmost importance and can be expedited by an accessible, respiratory specific monitor. Such a system would provide researchers with a streamlined tool for collecting information on AOP and other conditions. Moreover, integrating additional clinical metrics (e.g., patient treatment, condition) helps HCPs determine the most optimal treatments for improving their patient's outcome.

The present invention features a novel, highly stretchable, soft strain sensor that measures patient torso expansion and contraction from breathing (respiratory effort). In a previous work, it was demonstrated that this system can accurately measure both respiratory rate and volume using two, 1 cm long, sensors placed on the patient's abdomen and chest wall. This concept has been widely reported in the literature using RIP belts to monitor the circumferential change of the torso. However, the present device demonstrated that this type of respiratory information can still be extracted from a much smaller area of the torso. This overcomes the disadvantages of RIP belts (large size, proneness to motion artifacts) while maintaining a simple, high spatial resolution, method for measuring key ventilatory features. The present invention incorporates the strain sensor in a small, wireless patch to monitor respiratory waveform and rate. This first-generation device has undergone all the required verification and clinical validation testing and is currently undergoing FDA review.

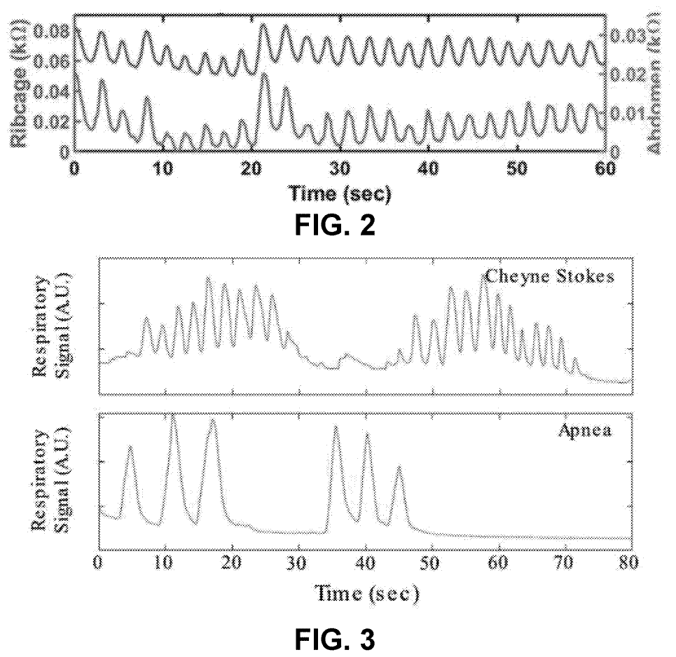

The soft strain sensor's size and monitoring capabilities makes it ideal for newborns. Similar to RIP, the soft sensors used by the RMS can monitor respiratory effort from both the abdomen and chest wall (FIG. 2) and can record patterns such as apnea, Cheyne-Stokes (FIG. 3), and paradoxical breathing in adults. Additionally, the highly conformal nature of the sensor makes measurements much more resilient to motion artifacts when compared to other traditional sensors such as RIP and impedance monitors. It was previously demonstrated the sensor's ability to continuously monitor for respiratory rate and depth even while the subject was performing extreme ambulatory movements such as running and sprinting. Moreover, the system was combined with state-of-the-art generative AI-driven bio-signal processing pipeline to accurately identify and reconstruct waveforms from motion artifacts as they occur.

AOP is primarily detected using standard vital sign monitors that heavily emphasize cardiovascular vital signs, which may not adequately capture apnea events. It was proposed that central, obstructive, and mixed apnea can be more accurately identified through a multimodal monitor integrating sensors with better respiratory monitoring capabilities and existing technologies (i.e., ECG) in a single device. Each type of apnea (central, obstructive, and mixed) presents distinct signs and symptoms that HCPs use for identification. Central apnea, stemming from depressed respiratory function, manifests as a lack of respiratory effort, evident by the absence of a respiratory waveform on monitors. Conversely, obstructive apnea results from airway obstruction, often indicated by attempts to breathe against the obstruction, paradoxical breathing patterns, or sternum retractions, detectable through measurements from an RIP belt.

Additionally, obstructive apnea is often accompanied by audible signs such as grunting or stridor. Mixed apnea is a combination of central and obstructive apnea and typically occurs in sequence. Apnea episodes may also coincide with changes in other vital signs, including bradycardia (decreased heart rate) and intermittent hypoxia (decreased blood oxygen levels), which can be measured with standard vital monitoring technology. Using targeted sensors for these specific signs paired with classical and advanced ML techniques enables better automatic identification of apnea based on the unique features and provide essential insights for AOP and its management.

The first objective was to create a fully integrated wireless apnea monitor for newborns capable of accurately identifying and distinguishing between central, obstructive, and mixed apnea events. This monitor consolidates different sensors into a single patch, with machine learning algorithms employed for apnea classification. User feedback informs the system design, and the monitor's performance is benchmarked against existing standards. Lastly, a mechanical stimulator for automatically terminating apnea was developed as well. The second objective was to establish a diverse and comprehensive cloud database containing high-quality, labeled AOP data. This database encompasses vital monitoring data alongside patient condition, treatment, and outcome information. Consequently, upon project completion, healthcare providers and researchers can retrospectively analyze the data to identify patterns and gain deeper insights into care and outcomes. Successful completion of these objectives helps enhance patient safety and care by providing a much-needed tool for both monitoring and research of AOP and advance understanding within the field.

A comprehensive and accurate apnea monitor has potential for enhancing patient safety and reducing the workload of HCPs. Additionally, better patient data not only improves care, but potentially reduces hospital stays, thereby easing the financial and emotional burden on families. Moreover, the increased accessibility of such a monitoring device can help address health disparities, particularly in minority communities where apnea occurs more frequently and is often underreported. A platform with user-friendly data collection capabilities can provide crucial cardiorespiratory information to better understand AOP and its associations with other conditions such as sepsis and NEC. This creates a comprehensive database of labeled information that can help advance the understanding of AOP.

There currently exists no integrated, multimodal, system for monitoring apnea in premature newborns. Thus, an objective is to combine five key sensors used for detecting apnea into a single wireless patch. The expected sensors are as follows. (1) custom strain sensor for monitoring respiratory effort. (2) electrocardiogram (ECG) sensors for monitoring heart rate. (3) Microphone for monitoring sound (i.e., grunting, stridor, etc.). (4) Pulse oximeter for monitoring blood oxygenation (5). And internal measurement units (IMUs) for measuring movement. In addition to incorporating the sensors listed above, the prototype also accounts for device footprint and battery life, two other key parameters for the study. An initial software was created to allow for easy data collection and storage onto the cloud, and a custom user interface is created for faster data labeling.

There are three main circuit boards designed. The first circuit is placed on the torso and contains all sensors, minus the pulse oximeter, for monitoring apnea. The microcontroller unit (MCU) on this circuit processes 7 analog and/or digital inputs from the sensors (4 from the strain sensor, 1 from the ECG, 1 from the microphone, and 1 from the IMU). The second circuit board is placed on the foot to monitor motion and blood oxygenation (pulse oximeter), and the last circuit is placed on the hand for monitoring motion. At this stage, battery life is maximized through firmware (controlling MCU cycle) and battery selection.

The hardware design focuses on size, safety, and sensor placement. The size of the device is determined during user interview, but current constraints are based on approved FDA devices for the NICU (4 cm×7 cm and weighs less than 20 grams). Low trauma adhesives (silicone based) are chosen for safety. Each sensor is connected to the circuit board via wire so that sensor placement can be optimized; this allows for more accurate and consistent placement across different newborn body sizes.

This is the first clinical study for collecting AOP data using the apnea prototype. To ensure high quality data collection, an iterative approach is used. Time series data is collected from premature newborns using both the apnea prototype and gold standard monitors. Motion artifacts and apnea are identified and labeled by HCPs. Any other relevant health information is also collected and uploaded, such as current treatment, treatment effectiveness, etc.

The study population is premature newborns born between gestational ages of 26 to 37 weeks; 10 days are allotted after birth to ensure appropriate skin integrity. Assuming a rate of 1 apnea episode every 2 hours, a 12-hour study in 20 patients provides at least 120 apneic events; this meets the target needed for ML models. Assuming a 50% factor for unforeseen circumstances, 30 patients are targeted for enrollment.

The premature newborns have standard hospital cardiorespiratory monitors attached from which the data is continuously collected. Additionally, an airflow monitor and audio video recording are used to help identify apneas. Each newborn has the apnea prototype placed based on the location determined previously. All recordings are collected for a duration of 12 hours.

The experimental team first agrees on the signs and symptoms of apnea from the cardiorespiratory, flow, and video data. The HCPs are blinded to the data and reviews and labels the data for apnea. Per the FDA guidance, apnea is classified as either type 1 (10 to 20 seconds) or type 2 (20 seconds or greater). A trained member reviews and labels the IMU data and video recording for motion. All labeling is done using the custom user interface to streamline the process. The data is reviewed as a team at set checkpoints to quickly identify potential problems; each review is done after 5 patients. The labeled apnea data is cross-examined for consistency, and the time-series data from each sensor is assessed for quality (SNR, noise, etc.) over the 12 hours. Additionally, preliminary features from the different apneas are verified. Any problems found are addressed immediately.

The labeled motion data is divided into a training set (70%) and test set (30%). Motion is often identified through a change in signal amplitude and frequency from the IMU [14], so a simple algorithm is developed to measure these two parameters in each IMU sensor component. The start and end time of each identified movement are recorded and compared to the labeled data; any values within 5 seconds are marked as the same event. The sensitivity and specificity are then assessed for a combination of different IMU sensors in a simple decision tree model. The Results are compared using the area under the curve (AUC) for the receiver operating characteristics (ROC) curve.

The team then focuses on connecting motion with apnea by looking for temporal correlation between spontaneous motion and apnea in the labeled dataset. According to literature, motion often precedes or occurs in conjunction with an apnea event. Thus, any events where motion occurred during 5 seconds before or after the apnea event are identified. A Monte Carlo simulation is then used to assess the probability of apnea and motion alignment. If a statistically significant relationship is found, then the data from the IMU is used to help identify apnea.

Data preparation and feature extraction is one of the most important steps for developing an accurate statistical ML model. The present invention concentrates on leveraging deep learning techniques on a larger data set. This dual strategy not only achieves maximal accuracy but also facilitates the discovery of novel insights within the domain. A major focus of this sub-aim is to identify clear features for classifying apnea. HCPs already have well-defined signs and symptoms they use that are informed from a physiological perspective. These signs and symptoms are used to help guide the selection of appropriate features in the ML models. Regarding ML, given the expected simplicity of classical models, several ML models are trained and assessed within a short timeframe with relatively low effort. Random Forest (RF) and Support Vector Machine (SVM) models are used. Moreover, methods to assess the overall importance of each feature are employed. This comprehensive approach allows us to understand the most important feature for classifying central and obstructive apnea.

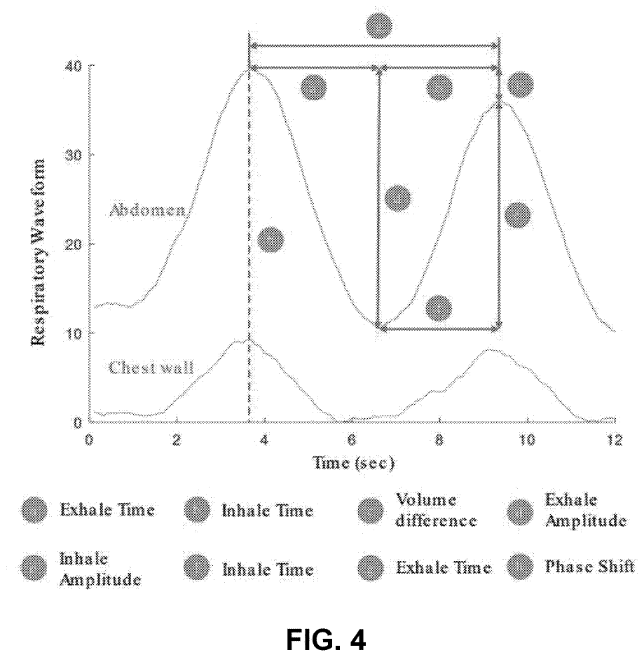

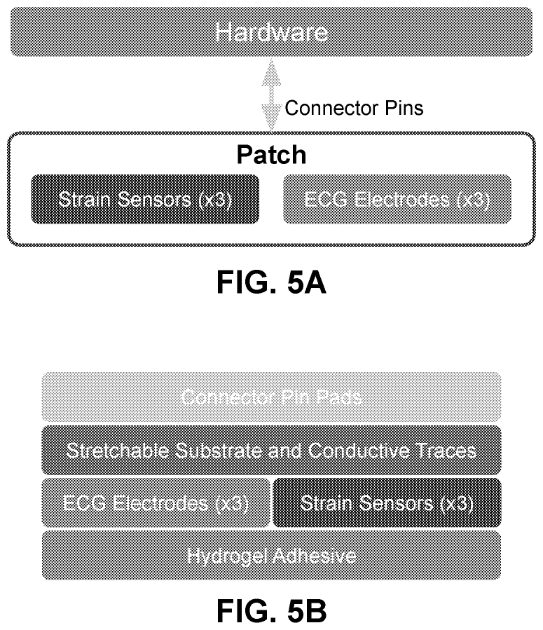

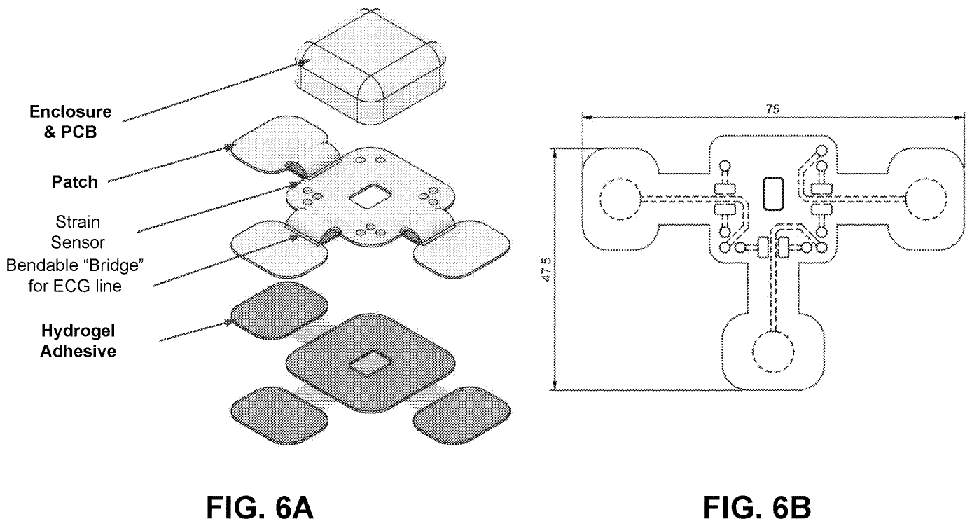

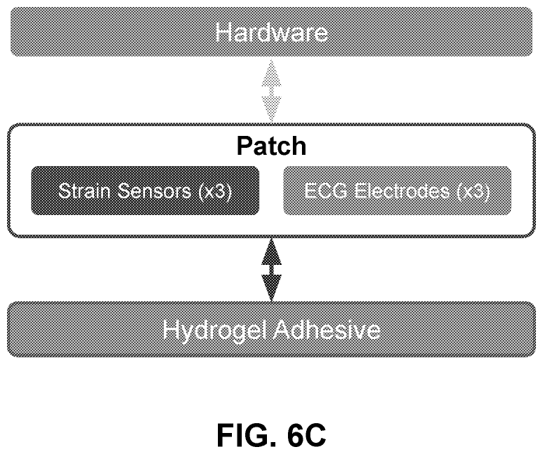

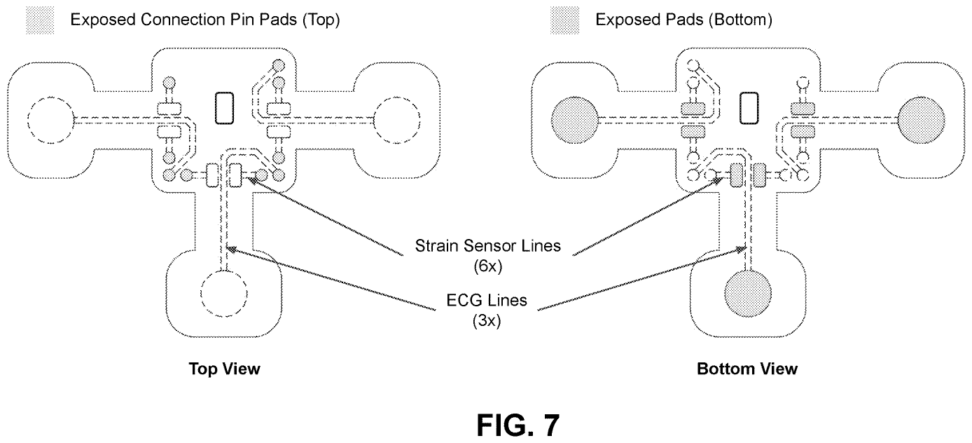

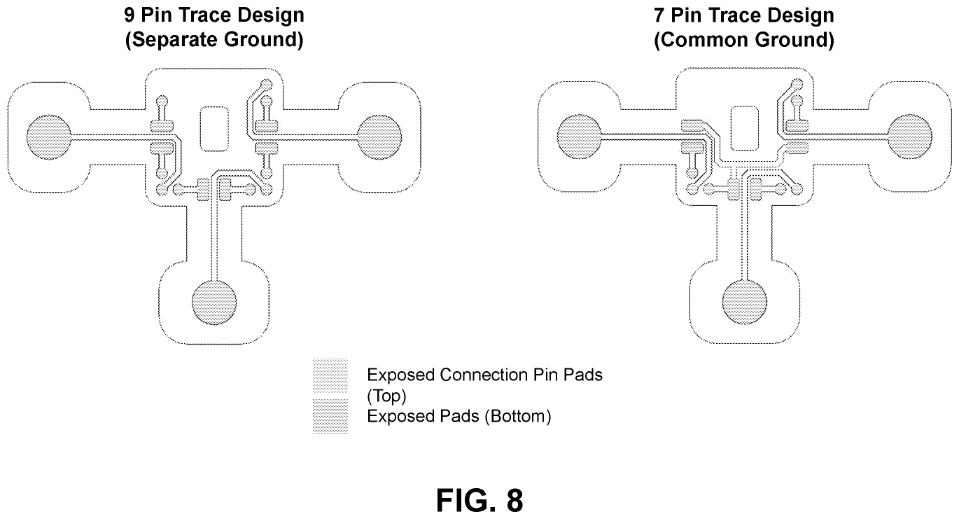

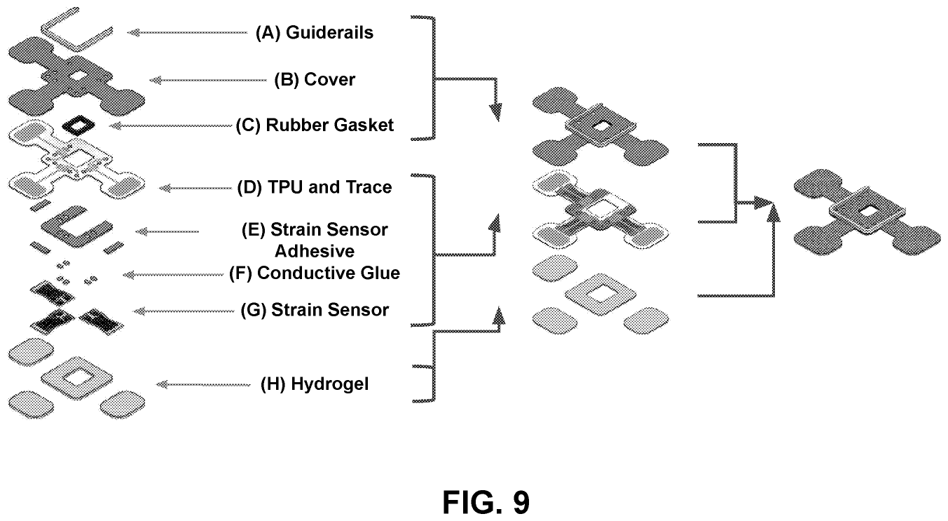

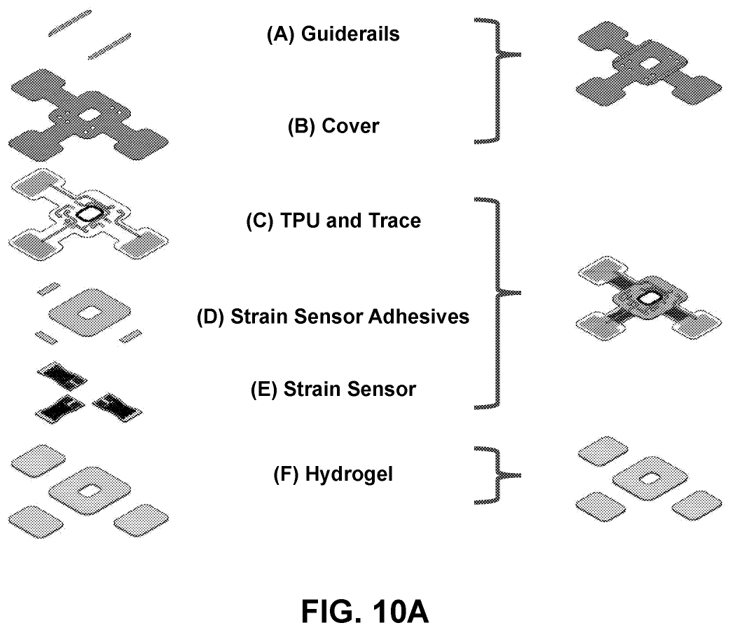

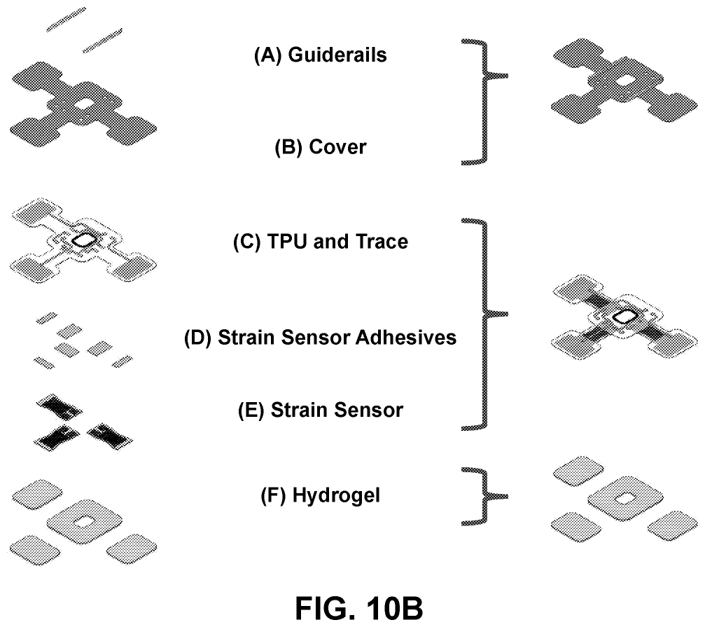

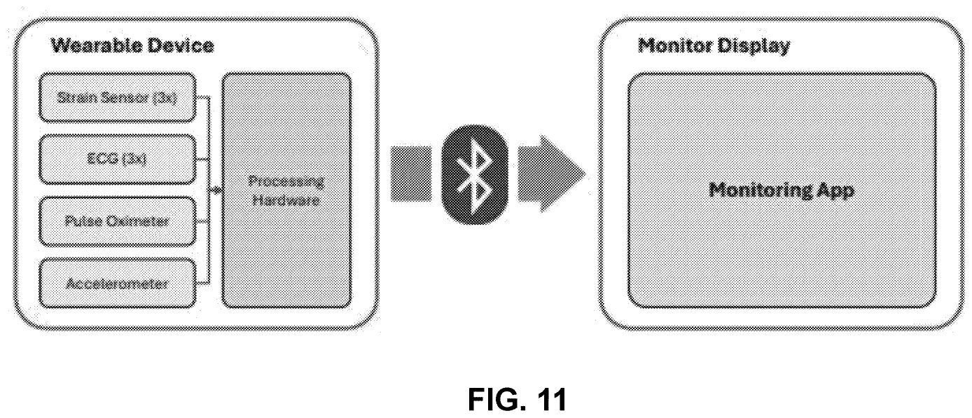

The approach follows a traditional ML development process. The first step is to review the dataset for quality and split the labeled apnea events into training (70%) and test (30%) cases. The data from the Makani Science apnea monitor is reviewed with HCPs to confirm the presence of clinically relevant signs and symptoms (i.e., paradoxical breathing during obstructive apnea); afterwards, specific features from each sensor signal are identified. FIG. 4 shows features that can be extracted from the strain sensor data. Multiple features are identified for each sign and symptoms. Once the features have been identified, algorithms are developed for extracting each feature. The sensor data is first filtered to remove noise and motion artifacts. Most of the anticipated features can be extracted using a robust peak detection algorithm. Other commonly used algorithms, such as the Fast Fourier Transform (FFT), are implemented to extract additional information. The sensitivity and specificity for the algorithm performance is also quantified as well.

All recorded data and extracted features are carefully reviewed and organized for their respective supervised machine learning (ML) models, starting with RF and SVM. Each model is constructed in Python utilizing the appropriate libraries, and their performance is assessed and compared using the AUC of the ROC. Following the initial model development, hyperparameter optimization is systematically carried out for all models. Central apnea is expected to initially exhibit higher sensitivity due to its distinctiveness from normal breathing. However, with appropriate feature selection, sensitivity for obstructive apnea should also be comparable. Mixed apnea is likely to be identified by detecting the proximity of two apnea events occurring close together.

Both RF and SVM models offer the ability to elucidate the contribution of each feature to the overall classification of apnea. This feature-important analysis is integrated into the output of each model, enabling us to identify the most influential features. Additionally, Gini Importance, t-SNE, and Principal Component Analysis (PCA) are separately applied to visualize and validate the contribution of these features. Once identified, new models are developed and evaluated using only the most influential features.

A diverse dataset is required for a robust ML algorithm. The goal of this sub-aim is to conduct additional studies to balance the current database gender and gestational age. The secondary objective of this aim is to verify the sensor's performance for a longer duration. The primary change is adjusting the study endpoint to balance gender and gestational age. Additionally, the duration of the study is increased to 48 hours. A sample size of 40 patients is targeted with a 50% buffer. The data is analyzed as they are collected, and a team review is performed every 10 subjects.

The data is split and organized between gender and age groups. Since this is a larger dataset, 50% of the data is reserved for training (if needed), while the other 50% is used for testing. Once the features have been organized, each model is evaluated across gender and gestational age. While no difference is expected between genders, as the physiology of newborns should be homogeneous, there may be some differences based on extreme gestational age.

ML models are built that can process and relate information from different modalities. It is posited that in apnea detection, the captured interactions between modalities are both supplementary and complementary, and can be used to improve performance, particularly in noisy/missing data scenarios. The prospective data collection can enable the identification of more robust, accurate, and missing/noisy data resilient techniques. In particular, three key aspects of modern multimodal ML are focused on: (1) Representation (how to represent data to exploit the complementarity and redundancy of multiple modalities measured by the sensors), (2) Fusion (to join information from these modalities to perform a prediction), and (3) Co-learning (to transfer knowledge between modalities, their representation, and their predictive models). With respect to representation, both joint representations are exploited (e.g., deep learning networks with attention mechanisms) and coordinated representations (e.g., minimizing cosine distance) methods to identify information that can be obtained in the absence of data. The primary choice is to use attention-based deep learning methods applied to time series data, which allow for performing representation and fusion in an end-to-end way.

Different techniques are investigated (e.g., temporal, time series transformer, etc.) with respect to both model performance and explainability. With respect to fusion, both model-agnostic (e.g., feature-based, hybrid fusion etc.) and model-based approaches (e.g., multiple kernel learning) are examined. In particular, the hybrid model-agnostic fusion mechanisms are of interest since they allow for predictions when one or more of the modalities are missing while capturing low-level interactions among the modalities. For co-learning, the concepts of co-training and transfer learning are exploited to improve robustness and missing/noisy data resiliency. Co-training is used to deal with disagreement between modalities by filtering out unreliable sensory data.

Feedback from 15 doctors and/or nurses is solicited after simulated use of the apnea monitor. First, a use related risk analysis (URRA) is completed to identify any major hazard from user error. Afterwards, the appropriate mitigations are proposed and tested by the end users (HCPs) in simulated scenarios. Each simulation is closely monitored, and the mitigation's effectiveness is evaluated. Additionally, end users are asked to provide feedback on the current design. The study result and feedback are documented and summarized in the product requirement document, and any suggested changes or additional features are incorporated in the final design.

By identifying the physical constraints and placements for the sensors, the framework for the PCB, adhesive, and case design is set. To minimize the risk of poor sensor performance, each sensor's design, location, and orientation closely match their counterpart. In parallel, the circuit is optimized for size and power. The first step is to choose a suitable MCU/Bluetooth chip with low power consumption. Afterwards, the circuit design is modified for energy efficiency (e.g., selecting low quiescent current amplifiers). Lastly, an appropriate battery (either rechargeable or disposable) is selected for duration. Based on past experience, a 30% to 50% reduction in size is expected, while being able to achieve the 2-week use period. The software and algorithm is also integrated together on the monitor.

Nurses' heavy workload can lead to delayed responses to apnea alarms, posing risks for neonates requiring immediate intervention to stimulate breathing. Integrating a stimulator system with the apnea monitor could enhance newborn safety and alleviate the burden on healthcare professionals. The Bluetooth and circuitry used in the prototype are combined with a mechanical transducer to provide the mechanical stimulation. The amplitude and vibration amplitude is controllable by a software application. The initial device has a stimulation frequency of 250 Hz for a duration of 10 seconds as these were effective parameters. To mitigate any risk of transferring too much mechanical energy to the newborn, a soft silicone buffer is placed between the device and the newborn. Finally, a closed loop system is programmed into the software application. The software is adjusted to activate the mechanical stimulator when apnea is detected. All components are verified benchtop with the appropriate verification protocol.

The following is another non-limiting example of the presently claimed invention.

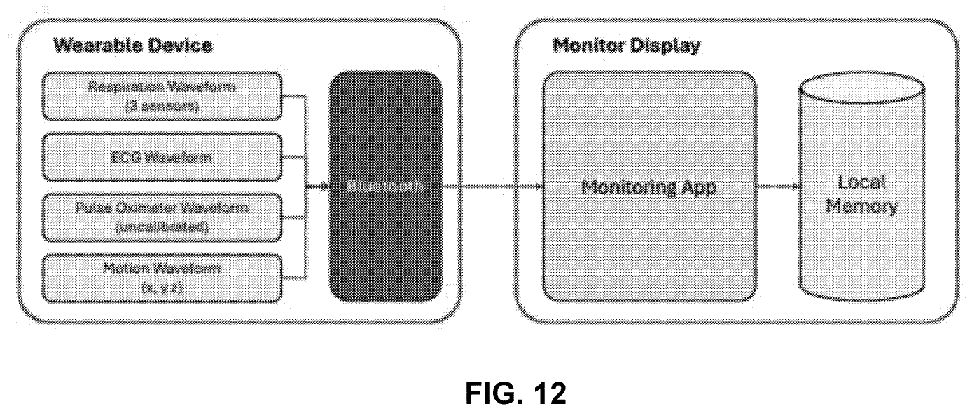

The Neonatal Monitoring System (NMS) is a non-invasive and wireless system intended to monitor and collect continuous vital sign information from neonatal patients. It records and displays respiratory, heart, and pulse oximeters, and motion waveforms. The sensor device of the present invention is composed of the following primary components described in FIG. 12. The NMS comprises a wireless device that contains multiple non-invasive sensors for vital sign monitoring. This device approximately measures 45 mm×65 mm×10 mm (1.8″×2.5″×0.4″) and is placed on the patient. The device is less than 100 g. The NMS further comprises a smart device that receives information from the wearable device for visual display and recording. The smart device could be a tablet or smart phone and contains a custom app for recording and displaying data. The NMS further comprises a custom app that processes and stores all vital sign data locally.

All data is recorded by the wearable device. Description of each sensor can be found below. The strain sensors are ultra soft silicone-based strain sensors that change in resistance when stretched. The sensors are used to measure the expansion and contraction of the torso and provide an analog of respiratory waveform. These sensors have been FDA cleared for monitoring respiratory rate in adults and have also been peer reviewed in multiple publications. The silicone used in manufacturing the strain sensors is safe for human skin. Three strain sensors are integrated into the wearable device.

ECG electrodes are custom printed by a medical device company that specializes in electrode design. A sensing hydrogel is used to interface the electrode with the body. Three ECG electrodes are integrated into the wearable device. The pulse oximeter uses a sensor that is integrated into the hardware. A clear window in the enclosure separates the pulse oximeter sensor and the skin of the patient. A single pulse oximeter sensor is integrated into the wearable device. The accelerometer is a sensor that is integrated into the hardware. The accelerometer does not require any access to the patient and measures the general motion of the hardware, which is used as an analog for patient motion. A single accelerometer is used on the board.

The final output of the NMS is timestamped waveforms for respiration, ECG, blood oxygenation, and motion saved as a comma separated file type. The following datasets are expected from the wearable device: Respiration Waveform (Strain Sensor 1), Respiration Waveform (Strain Sensor 2), Respiration Waveform (Strain Sensor 3), ECG Waveform (Lead 1), Blood Oxygenation Waveform, Motion (x-direction), Motion (y-direction), Motion (z-direction).

The wearable device has the following modes: Standby Mode, which is a low power mode where the device is paired but monitoring has not started; Pairing Mode, where the monitor is actively pairing with the device; Monitoring Mode, where the device is actively monitoring the patient; and Charging Mode, where the device is not in use and charging. The app has the following modes: Standby mode, where no monitoring is occurring and users can actively access past data and download; Pairing Mode, where the app is actively pairing with the device; and Monitoring Mode, where the app is actively reading data from the wearable device.

The wearable device and monitor communicates via Bluetooth Low energy using a Bluetooth module. FIG. 12 shows the anticipated flow of data from the wearable device to the monitor. No protected health information is recorded by the NMS.

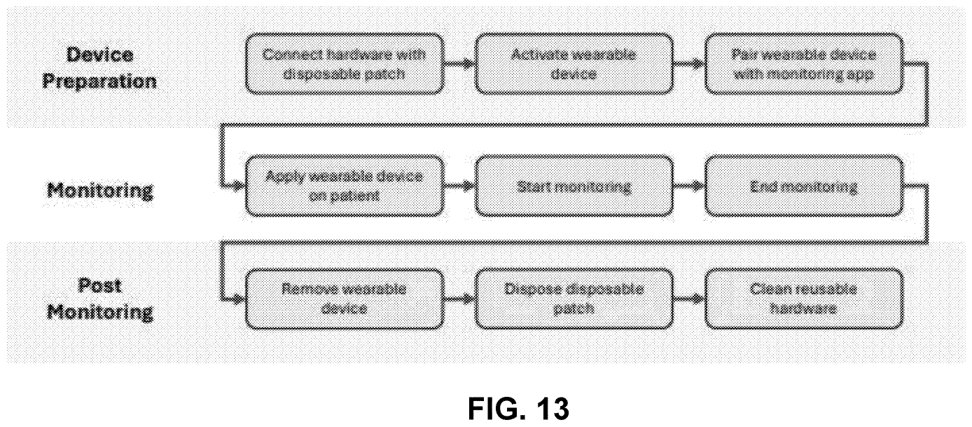

The wearable device is intended to be applied on the torso of the neonate for monitoring up to 24 hours. The primary location of the application is below the sternum with each lead of the electrode applied towards the peripheral of the torso in a “T” shape formation, with the bottom of the T pointing towards the navel. It should be noted that placement location may vary depending on availability of space on the skin. The wearable hardware is in a low power state until it is activated through motion. Once activated, Bluetooth pairing occurs automatically after a unique QR code on the reusable hardware that is scanned by the monitoring. Once paired, the wearable device can then be applied and monitoring can start. FIG. 13 shows an example of how the NMS is prepared and applied onto a patient for monitoring.

The NMS has three main components and two physical interfaces. The NMS comprises a wearable device that is adhered to the patient. It is composed of two sub-components: a reusable hardware and a disposable patch. The NMS further comprises a monitoring display, comprising a smart device for recording and displaying data from the wearable device. The NMS further comprises a monitoring app for reading and processing the data from the wearable device. The NMS further comprises a reusable hardware/disposable patch interface between the reusable hardware and disposable patch. The NMA further comprises a wearable device/body interface between the wearable device and the body.

The wearable device contains all the sensors described above. It is an assembly of two sub-components: (1) a reusable hardware component that contains a printed circuit board (PCB) and battery within a waterproof enclosure and (2) a disposable patch that contains strain sensors and ECG pads. The reusable hardware and disposable patch are mechanically integrated using guiderails.

A soft silicone enclosure is used to cover the hardware and battery of the reusable hardware. The entire enclosure plus the base is waterproof and cleanable using alcohol. A soft enclosure, in addition to rounded edges, is important for reducing the chances of pressure injuries. A lithium polymer rechargeable battery is integrated with hardware. This component is rechargeable through the POGO pin interface at the bottom of the enclosure. The PCB contains the microcontroller and Bluetooth module for communicating with the monitoring display. It also has the pulse oximeter component and accelerometer integrated into the circuitry. The PCB is fabricated using conventional processes and is RoHS compliant. This component is not intended for direct contact with the newborn. A stiff ABS base is used to support the hardware and integrate with the disposable patch via a mechanical rail mechanism. POGO pins through the base are used to establish electrical connection with the patch. The base is also waterproof and cleanable using alcohol. Rails used to mechanically lock the reusable hardware in place. A medical grade adhesive adhered to the top of the stack provides a clean finish and increases robustness. A flexible layer with conductive traces connects the three ECG electrodes and three strain sensors from their respective location to the POGO pins for the reusable hardware. The flexible circuit substrate is TPU with stretchable silver trace printed on both sides. An electrically conductive adhesive both mechanically and electrically connects the strain sensor to the flexible circuit layer. Three strain sensors are integrated between the flexible circuit layer and the hydrogel. A sensing hydrogel is used to adhere the sensor to the patient and doubles as the sensing gel for the ECG.

The monitoring display is a smart device that receives information from the wearable device via Bluetooth. The smart device is an iPad™ or iPhone™. The Apple™ ecosystem has strong security that is leveraged. A custom app communicates exclusively with the wearable device. The primary function of the app is to receive and record the continuous waveform from the wearable device and display real time waveforms.

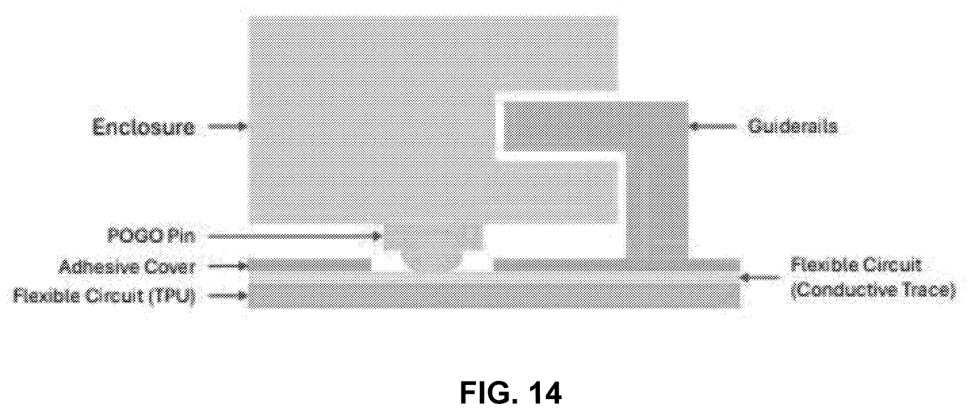

The reusable hardware and disposable patch interfaces both mechanically and electrically. The mechanical interface uses guide rails on the disposable patch that slot into corresponding features on the reusable hardware to ensure stable alignment and secure attachment. The electrical interface uses POGO pins in the reusable hardware that contact exposed electrical pads on the surface of the disposable patch. FIG. 14 shows a side profile of the interface. The wearable device is adhered to the body using a skin-safe conductive hydrogel. This hydrogel serves as both a mechanical and electrical interface with the body. Mechanically, it secures the device to the skin and provides anchor points that enable pulling and stretching of the strain sensor. Electrically, it establishes a conductive path between the ECG electrode and the body.

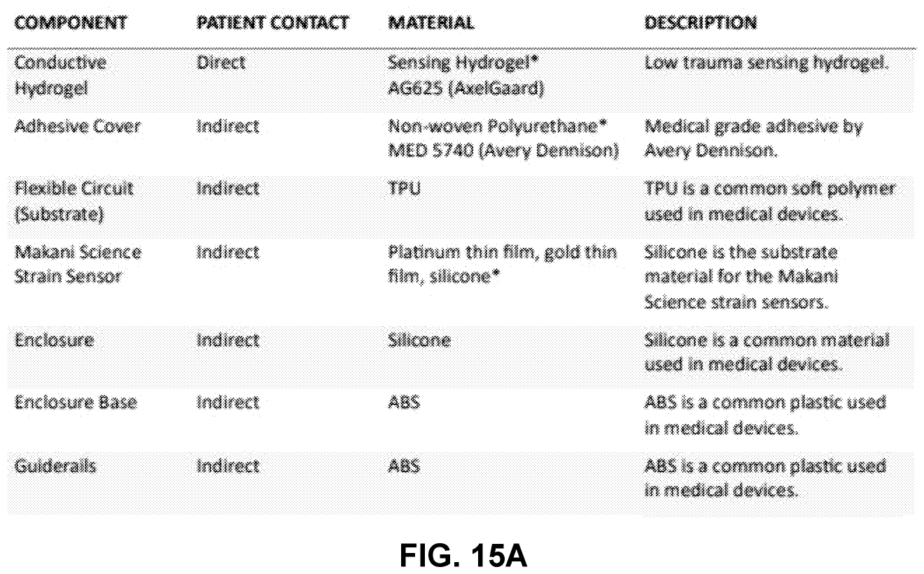

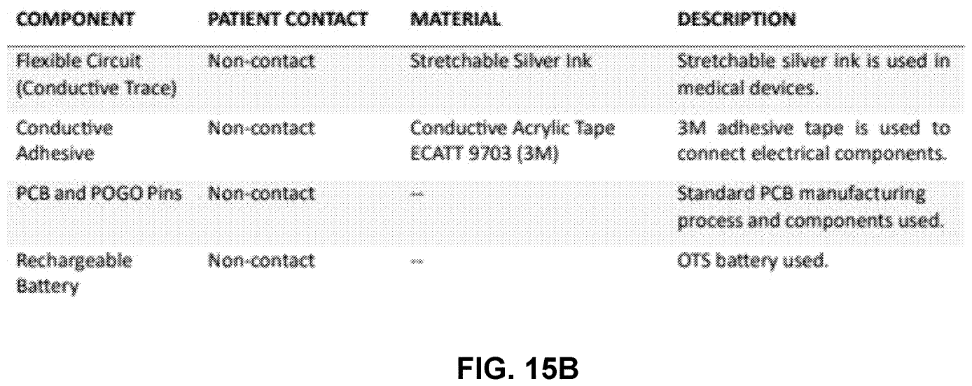

FIG. 15A shows a list of all components that may contact the patient and their material. Direct patient contact means the material is intended to contact the patient; indirect patient contact means the material is not intended to contact with the patient, but contact may happen. FIG. 15B shows a list of components that are not intended to contact the patient.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

The reference numbers recited in the below claims are solely for ease of examination of this patent application, and are exemplary, and are not intended in any way to limit the scope of the claims to the particular features having the corresponding reference numbers in the drawings.

What is claimed is:

1. A system (100) for respiratory event prediction and detection in a patient, the system (100) comprising:
a. a multimodal sensor system (110) comprising a plurality of sensor devices disposed on or proximate to a body of the patient, configured to generate one or more output signals, wherein the plurality of sensor devices comprises a plurality of mechanical sensors (112) disposed on a torso of the patient configured to measure one or more mechanical properties of the patient and generate one or more respiratory signals based on the one or more mechanical properties, wherein the one or more output signals comprises the one or more respiratory signals, wherein the plurality of mechanical sensors (112) comprise soft electronic strain sensors; and
b. a computing device (120) communicatively coupled to the multimodal sensor system (110), comprising a predictive model (121) configured to accept the one or more output signals and generate an output comprising an identification of a respiratory event, a prediction of a future respiratory event, or a combination thereof.

2. The system (100) of claim 1, wherein the plurality of mechanical sensors (112) further comprise motion sensors, internal measurement units (IMUs), or a combination thereof.

3. The system (100) of claim 1, wherein the plurality of sensor devices further comprise:
a. one or more electrical sensors configured to measure one or more electrical signals;
b. one or more acoustic sensors configured to measure one or more acoustic signals;
c. one or more light sensors configured to measure one or more light-based signals; and
d. one or more chemical sensors configured to measure one or more chemical signals.

4. The system (100) of claim 3, wherein the predictive model (121) is further configured to filter one or more false positives from the output based on the one or more electrical signals, the one or more acoustic signals, the one or more light-based signals, the one or more chemical signals, or a combination thereof.

5. The system (100) of claim 3, wherein the plurality of mechanical sensors (112) further comprise accelerometers configured to measure one or more internal motion signals, one or more external motion signals, or a combination thereof, wherein the predictive model (121) is further configured to filter one or more motion artifacts from the output based on the one or more internal motion signals, the one or more external motion signals, or the combination thereof.

6. The system (100) of claim 1 further comprising a stimulator device (130) communicatively coupled to the computing device (120) and disposed on or proximate to the body of the patient, configured to apply stimulation to the body of the patient in response to the identification of the respiratory event, the prediction of the future respiratory event, or the combination thereof such that the respiratory event, the future respiratory event, or a combination thereof is interrupted.

7. The system (100) of claim 6, wherein the stimulation applied by the stimulator device (130) comprises vibration, heat, sound, electrical current, movement, or a combination thereof.

8. The system (100) of claim 1, wherein the respiratory event, the future respiratory event, or the combination thereof comprises apnea, snoring, external obstruction of breathing, internal obstruction of breathing, Sudden Infant Death Syndrome (SIDS), or a combination thereof.

9. The system (100) of claim 1, wherein the predictive model (121) comprises an algorithm, a machine learning model, or a combination thereof.

10. A system (100) for apneic event prediction and detection in a patient, the system (100) comprising:
a. a multimodal sensor system (110) comprising a plurality of sensor devices disposed on or proximate to a body of the patient, configured to generate one or more output signals, wherein the plurality of sensor devices comprises a plurality of mechanical sensors (112) disposed on a torso of the patient configured to measure one or more mechanical properties of the patient and generate one or more respiratory signals based on the one or more mechanical properties, wherein the one or more output signals comprises the one or more respiratory signals, wherein the plurality of mechanical sensors (112) comprise soft electronic strain sensors;
b. a computing device (120) communicatively coupled to the multimodal sensor system (110), comprising a predictive model (121) configured to:
i. accept the one or more output signals;
ii. generate an output comprising an identification of an apneic event, a prediction of a future apneic event, or a combination thereof;
iii. categorize the apneic event, the future apneic event, or a combination thereof as central apnea, obstructive apnea, or a combination thereof; and
iv. transmit a signal to a stimulator device (130) in response to the identification of the apneic event, the prediction of the future apneic event, or the combination thereof; and
c. the stimulator device (130) communicatively coupled to the computing device (120) and disposed on the body of the patient, configured to apply stimulation to the body of the patient in response to the identification of the apneic event, the prediction of the future apneic event, or the combination thereof such that the apneic event, the future apneic event, or the combination thereof is interrupted.

11. The system (100) of claim 10, wherein the plurality of mechanical sensors (112) further comprise motion sensors, internal measurement units (IMUs), or a combination thereof.

12. The system (100) of claim 10, wherein the plurality of sensor devices further comprise:
a. one or more electrocardiogram (ECG) electrodes configured to measure one or more electrical activity signals;
b. one or more microphones configured to measure one or more sound signals;
c. one or more internal measurement units (IMUs) configured to measure one or more internal motion signals;
d. one or more pulse oximeter devices configured to measure one or more blood oxygenation signals; and
e. one or more motion sensors configured to measure one or more external motion signals.

13. The system (100) of claim 12, wherein the predictive model (121) is further configured to filter one or more false positives from the output based on the one or more electrical activity signals, the one or more sound signals, the one or more blood oxygenation signals, or a combination thereof.

14. The system (100) of claim 12, wherein the predictive model (121) is further configured to filter one or more motion artifacts from the output based on the one or more internal motion signals, the one or more external motion signals, or a combination thereof.

15. The system (100) of claim 10, wherein the stimulation applied by the stimulator device (130) comprises vibration, heat, sound, electrical current, or a combination thereof.

16. The system (100) of claim 10, wherein the future apneic event comprises apnea prematurity.

17. The system (100) of claim 10, wherein categorizing the apneic event, the future apneic event, or the combination thereof comprises determining whether the apneic event, the future apneic event, or the combination thereof comprises central apnea, obstructive apnea, or a combination thereof.

18. The system (100) of claim 10, wherein the predictive model (121) comprises an algorithm, a machine learning model, or a combination thereof.

19. A system (100) for apneic event prediction and detection in an infant patient, the system (100) comprising:
a. a multimodal sensor system (110) comprising a plurality of sensor devices disposed on or proximate to a body of the infant patient, configured to generate one or more output signals, wherein the plurality of sensor devices comprises a plurality of mechanical sensors (112) disposed on a torso of the infant patient configured to measure one or more mechanical properties of the infant patient and generate one or more respiratory signals based on the one or more mechanical properties, wherein the one or more output signals comprises the one or more respiratory signals, wherein the plurality of mechanical sensors (112) further comprise soft electronic strain sensors;
b. a computing device (120) communicatively coupled to the multimodal sensor system (110), comprising a processor (122) configured to execute computer-readable instructions and a memory component (124) operatively coupled to the processor (122), comprising:
i. a predictive model (121) configured to accept the one or more output signals and generate an output comprising an identification of an apneic event, a prediction of a future apneic event, or a combination thereof; and
ii. computer-readable instructions for:
A. inputting the one or more output signals into the predictive model (121) such that the predictive model (121) generates the output;
B. determining, based on the output, whether the apneic event, the future apneic event, or the combination thereof comprises central apnea, obstructive apnea, or a combination thereof; and
C. transmitting a signal to a stimulator device (130) in response to the identification of the apneic event, the prediction of the future apneic event, or the combination thereof; and
c. the stimulator device (130) communicatively coupled to the computing device (120) and disposed on the body of the infant patient, configured to apply stimulation to the body of the infant patient in response to the signal from the computing device (120) such that the apneic event, the future apneic event, or the combination thereof is interrupted.

20. The system (100) of claim 19, wherein the predictive model (121) comprises an algorithm, a machine learning model, or a combination thereof.
